(19) United States
(12) Patent Application Publication
(10) Pub. No.: US 2023/0284999 A1
(43) Pub. Date: Sep. 14, 2023

(54) Integrated Electrodiagnostic and Ultrasound Device

(71) Applicant: Cadwell Laboratories, Inc., Kennewick, WA (US)

(72) Inventors: Michal Holub, JR., Richland, WA (US); Abram Gardner, San Clemente, CA (US)

(21) Appl. No.: 18/183,350

(22) Filed: Mar. 14, 2023

Related U.S. Application Data
(60) Provisional application No. 63/319,654, filed on Mar. 14, 2022.

(51) Int. Cl. A61B 8/00; A61B 5/25

(52) U.S. Cl. CPC A61B 8/4444; A61B 5/25

(57) ABSTRACT

A device for electrically stimulating, and measuring electrical responses of, an internal organ of a patient while obtaining real time images of the internal organ, the device having an ultrasound probe; one or more electrodes connected to the ultrasound probe for electrically stimulating the organ and measuring responses from the organ, wherein the probe is placed at a location on the patient's body proximate the internal organ for obtaining the real time images of the organ; and a stimulator connected to the one or more electrodes for providing electric current of a predefined amplitude for stimulating the internal organ.

### CROSS-REFERENCE

The present specification relies on U.S. Patent Provisional No. 63/319,654, titled “Integrated Electrodiagnostic and Ultrasound Device” and filed on Mar. 14, 2022. The above-mentioned application is herein incorporated by reference in its entirety.

### FIELD

The present specification relates to diagnostic systems. More particularly, the present specification relates to electrodiagnostic and ultrasound technology.

### BACKGROUND

An ultrasound is an imaging test that uses sound waves to create a picture, also known as a sonogram, of organs, tissues, and other structures within the body. An ultrasound can also show parts of the body in motion, such as a heart beating or blood flowing through blood vessels. Conventional ultrasound machines comprise a computer console, video monitor, and an attached transducer, also known as a probe. The transducer is a small hand-held device which is placed on an area of a patient's body that needs to be examined. During an ultrasound imaging procedure, a technologist applies a small amount of gel on an area of a patient's body that needs to be examined and places the transducer on the area. The gel allows sound waves to travel back and forth between the probe and the area under examination. The transducer transmits inaudible, high-frequency sound waves into the body and listens for the returning echoes. The ultrasound image is immediately visible on a video monitor. The processor generates an image based on the loudness (amplitude), pitch (frequency), and time it takes for the ultrasound signal to return to the probe.

Electrodiagnosis involves the use of electrophysiological methods, such as, but not limited to, electroencephalography (EEG), electromyography (EMG), and evoked potentials, to diagnose the functional integrity of certain neural structures (e.g., nerves, spinal cord and parts of the brain) to assess disease states and determine potential therapy or treatment. Generally, electrodiagnostic procedures may involve a large number of electrodes coupled to the human body. For example, in an EEG procedure, electrodes are used to record and monitor the electrical activity corresponding to various parts of the brain for detection and treatment of various ailments such as epilepsy, sleep disorders and coma. Each of these electrodes is coupled to a wire lead which, in turn, is connected to a control unit adapted to receive and transmit electrical signals. The wire leads are usually coupled with standard connectors comprising a plug which fits into a corresponding receptacle for connecting with the control unit of monitoring equipment, such as, for example, EMG monitoring equipment. The electrical activity pattern captured by various electrodes is analyzed using standard algorithms to localize or spot the portion of brain or tissue which is responsible for causing the specific ailment.

During neuromuscular or intraoperative neuromonitoring procedures, the region of interest in a patient's body is preferably localized to ensure obtaining an optimal recording from the region, or for stimulating the region. Hence, there is need for a device that enables simultaneous visualization of a patient's internal organs or tissue while the electrical activity of the organs or tissue is being recorded, or while the organs or tissue are being stimulated electrically. There is also a need for a device that allows a clinician/doctor to record the electrical activity produced by the patient's organs from within the region of interest, electrically stimulate the organs within the region of interest, and to simultaneously observe the patient's internal organs during stimulation.

### SUMMARY

The following embodiments and aspects thereof are described and illustrated in conjunction with systems, tools and methods, which are meant to be exemplary and illustrative, and not limiting in scope. The present application discloses numerous embodiments.

The present specification discloses a device for electrically stimulating an internal organ of a patient while obtaining real time images of the internal organ, the device comprising: an ultrasound probe; and one or more electrodes coupled to the ultrasound probe for electrically stimulating the organ, wherein the probe is configured to be placed at a location on the patient's body proximate the internal organ for obtaining the real time images of the organ.

The present specification also discloses a device for recording electrical activity of an internal organ of a patient while obtaining real time images of the internal organ, the device comprising: an ultrasound probe; and one or more electrodes coupled to the ultrasound probe for electrically stimulating the organ, wherein the probe is placed at a location on the patient's body proximate the internal organ for obtaining the real time images of the organ.

In embodiments, the present specification is directed toward a probe configured to direct an electrical stimulation to an internal organ of a patient while concurrently directing ultrasound energy to said internal organ of the patient, wherein the handheld probe is in electrical communication with a pulse train generator, an ultrasound wave generator, at least one processor configured to process reflected ultrasound waves and a display, the handheld probe comprising: a housing body; an ultrasound probe positioned within the housing body, wherein the ultrasound probe comprises a plurality of transmitting elements and a plurality of receiving elements; and one or more electrodes coupled to an exterior of the housing body, wherein each of the one or more electrodes is positioned such that a periphery of each of the one or more electrodes is positioned less than 2 mm from a periphery of each of the plurality of receiving elements.

Optionally, the pulse train generator is configured to provide an electric current of a predefined amplitude for stimulating the internal organ.

Optionally, the one or more electrodes are single-use disposable electrodes.

Optionally, the one or more single-use electrodes are coupled with the housing body using adhesive or a molded housing.

Optionally, the one or more electrodes are reusable electrodes.

Optionally, the one or more reusable electrodes are coupled with the housing body using adhesive or a molded housing.

Optionally, the one or more electrodes are coupled to the pulse train generator using connecting wires having a thickness ranging from 1 AWG to 40 AWG, and preferably 10 AWG to 28 AWG.

Optionally, the connecting wires comprise touchproof or DIN connecting wires.

Optionally, a distance between the one or more electrodes is in a range of 10 mm to 25 mm.

Optionally, the ultrasound probe is coupled to an ultrasound machine comprising a display screen and a control board.

Optionally, the probe further comprises an amplifier coupled to the one or more electrodes and configured to process electrical activity of the internal organ.

In some embodiments, the present specification is directed towards a probe configured to direct an electrical stimulation to an internal organ of a patient while concurrently directing ultrasound energy to said internal organ of the patient, wherein the handheld probe is in electrical communication with a pulse train generator, an ultrasound wave generator, at least one processor configured to process reflected ultrasound waves and a display, the handheld probe comprising: a housing body; an ultrasound probe positioned within the housing body, wherein the ultrasound probe comprises a plurality of transmitting elements and a plurality of receiving elements; and one or more electrodes coupled to an exterior of the housing body, wherein each of the one or more electrodes is positioned such that a periphery of each of the one or more electrodes is positioned 2 mm or more from a periphery of each of the plurality of receiving elements.

Optionally, the pulse train generator is configured to provide an electric current of a predefined amplitude for stimulating the internal organ.

Optionally, the one or more electrodes are single-use disposable electrodes.

Optionally, the one or more single-use electrodes are coupled with the housing body using adhesive or a molded housing.

Optionally, the one or more electrodes are reusable electrodes.

Optionally, the one or more reusable electrodes are coupled with the housing body using adhesive or a molded housing.

Optionally, the one or more electrodes are coupled to the pulse train generator using connecting wires having a thickness ranging from 1 AWG to 40 AWG, and preferably 10 AWG to 28 AWG.

Optionally, the connecting wires comprise touchproof or DIN connecting wires.

Optionally, a distance between the one or more electrodes is in a range of 10 mm to 25 mm.

Optionally, the ultrasound probe is coupled to an ultrasound machine comprising a display screen and a control board.

Optionally, the probe further comprises an amplifier coupled to the one or more electrodes and configured to process electrical activity of the internal organ.

The aforementioned and other embodiments of the present specification shall be described in greater depth in the drawings and detailed description provided below.

### BRIEF DESCRIPTION OF THE DRAWINGS

The accompanying drawings illustrate various embodiments of systems, methods, and embodiments of various other aspects of the disclosure. Any person with ordinary skills in the art will appreciate that the illustrated element boundaries (e.g. boxes, groups of boxes, or other shapes) in the figures represent one example of the boundaries. It may be that in some examples one element may be designed as multiple elements or that multiple elements may be designed as one element. In some examples, an element shown as an internal component of one element may be implemented as an external component in another and vice versa. Furthermore, elements may not be drawn to scale. Non-limiting and non-exhaustive descriptions are described with reference to the following drawings. The components in the figures are not necessarily to scale, emphasis instead being placed upon illustrating principles.

FIG. 1A is an illustration showing two electrodes mounted on a same side of an ultrasound probe, in accordance with an embodiment of the present specification;

FIG. 1B is an illustration showing two electrodes mounted on different or opposing sides of an ultrasound probe, in accordance with an embodiment of the present specification;

FIG. 1C illustrates two electrodes mounted on a side of an ultrasound probe such that the electrodes are positioned away from an array of ultrasound elements within the probe, in accordance with an embodiment of the present specification;

FIG. 1D illustrates exemplary extension cables that may be used with the connecting wires shown in FIGS. 1A-1C, in accordance with some embodiments of the present specification;

FIG. 1E illustrates electrodes placed on a same side of an ultrasound probe, such that images of the electrodes are visible in the ultrasound image obtained via the probe, in accordance with an embodiment of the present specification;

FIG. 1F illustrates electrodes placed on opposite sides of an ultrasound probe, such that images of the electrodes are visible in the ultrasound image obtained via the probe, in accordance with an embodiment of the present specification;

FIG. 1G illustrates electrodes placed on a same side of an ultrasound probe, such that there is no interference between the electrodes and the signal of the probe and wherein the electrodes are placed within a channel molded on the probe body, in accordance with an embodiment of the present specification;

FIG. 1H illustrates electrodes placed on opposite sides of an ultrasound probe, such that there is no interference between the electrodes and the signal of the probe and wherein the electrodes are placed within a channel molded on the probe body, in accordance with an embodiment of the present specification;

FIG. 1I illustrates electrodes placed on a same side of an ultrasound probe, such that images of the electrodes are visible in the ultrasound image obtained via the probe and wherein the electrodes are placed within a channel molded on the probe body, in accordance with an embodiment of the present specification;

FIG. 1J illustrates electrodes placed on opposite sides of an ultrasound probe, such that images of the electrodes are visible in the ultrasound image obtained via the probe and wherein the electrodes are placed within a channel molded on the probe body, in accordance with an embodiment of the present specification;

FIG. 2A is an illustration showing an ultrasound probe covered using a sterile cover and with two electrodes mounted on the same side of the ultrasound probe, in accordance with an embodiment of the present specification;

FIG. 2B an illustration showing an ultrasound probe covered using a sterile cover and with two electrodes mounted on different sides of the ultrasound probe, in accordance with an embodiment of the present specification;

FIG. 2C is an illustration of a mechanical attachment carrying a pair of electrodes that may be removably attached to an ultrasound probe, in accordance with an embodiment of the present specification;

FIG. 2D illustrates a plurality of electrodes including an adhesive back for attachment with an ultrasound probe, in accordance with an embodiment of the present specification;

FIG. 3A illustrates electrodes coupled with an ultrasound probe, wherein the electrodes are attached to a connector, in accordance with an embodiment of the present specification;

FIG. 3B illustrates the electrodes shown in FIG. 3A connected, via the connector, to an amplifier, in accordance with an embodiment of the present specification;

FIG. 3C illustrates electrodes coupled with an ultrasound probe, wherein the electrodes are connected to a stimulator, in accordance with an embodiment of the present specification;

FIG. 3D illustrates the electrodes shown in FIG. 3C connected, via the stimulator, to an amplifier, in accordance with an embodiment of the present specification;

FIG. 4 is a graphical user interface (GUI) on a display showing waveforms recorded by an ultrasound probe being used in conjunction with recording/stimulating electrodes placed on a patient's arm, in accordance with an embodiment of the present specification;

FIG. 5A illustrates electrodes coupled with an ultrasound probe, wherein the electrodes are connected to a stimulator, in accordance with an embodiment of the present specification;

FIG. 5B is an ultrasound image of a patient's internal organ obtained using the ultrasound probe coupled with electrodes shown in FIG. 5A;

FIG. 5C is an ultrasound image of a patient's internal organ obtained using the ultrasound probe coupled with electrodes having thin connecting wires, in accordance with an embodiment of the present specification;

FIG. 5D illustrates connecting wires of varying thicknesses that may be used with electrodes coupled with an ultrasound probe, in accordance with embodiments of the present specification; and

FIG. 5E illustrates a plurality of differently sized wires that may be used with electrodes coupled with an ultrasound probe, in accordance with embodiments of the present specification.

### DETAILED DESCRIPTION

In various embodiments, the present specification describes devices comprising ultrasound diagnostic equipment with electrodes which are capable of recording electrical activity within a patient's organ and or stimulating the organ. The devices enable providing electrical stimulation to desired portions of the organ, thus providing an efficient treatment/diagnostic tool. In embodiments, the present specification describes a device that is configured to enable the use of electrodiagnostic technology with ultrasound technology for simultaneously viewing a patient's internal organs via an ultrasound probe, recording electrical activity of the organs via electrodes coupled to the probe, and optionally, electrically stimulating the organs via the electrodes.

The use of ultrasound technology enables a clinician/doctor to visualize a patient's anatomical structure and localize a region of interest. Electrodes combined with an ultrasound probe allow the clinician/doctor to record the electrical activity produced by the patient's organs from within the region of interest, electrically stimulate the organs within the region of interest, and simultaneously observe the patient's internal organs during stimulation and while the organs are functioning.

Hence, the device of the present specification eliminates the need for switching between an ultrasound probe and electrodiagnostic equipment during neuromuscular or intraoperative neuromonitoring procedures, thereby providing a more efficient workflow as well as improved functionality. Optionally, the position of electrodes coupled with a patient's body is continuously monitored/confirmed during the procedures.

The present specification is directed towards multiple embodiments. The following disclosure is provided in order to enable a person having ordinary skill in the art to practice the invention. Language used in this specification should not be interpreted as a general disavowal of any one specific embodiment or used to limit the claims beyond the meaning of the terms used therein. The general principles defined herein may be applied to other embodiments and applications without departing from the spirit and scope of the invention. Also, the terminology and phraseology used is for the purpose of describing exemplary embodiments and should not be considered limiting. Thus, the present invention is to be accorded the widest scope encompassing numerous alternatives, modifications and equivalents consistent with the principles and features disclosed. For purpose of clarity, details relating to technical material that is known in the technical fields related to the invention have not been described in detail so as not to unnecessarily obscure the present invention.

In various embodiments, a computing device/processor includes an input/output controller, at least one communications interface, and a system memory. The system memory includes at least one random access memory (RAM) and at least one read-only memory (ROM). These elements are in communication with a central processing unit (CPU) to enable operation of the computing device. In various embodiments, the computing device/processor may be a conventional standalone computer or alternatively, the functions of the computing device may be distributed across multiple computer systems and architectures.

In some embodiments, execution of a plurality of sequences of programmatic instructions or code enable or cause the CPU of the computing device/processor to perform various functions and processes. In alternate embodiments, hard-wired circuitry may be used in place of, or in combination with, software instructions for implementation of the processes of systems and methods described in this application. Thus, the systems and methods described are not limited to any specific combination of hardware and software.

In the description and claims of the application, each of the words “comprise”, “include”, “have”, “contain”, and forms thereof, are not necessarily limited to members in a list with which the words may be associated. Thus, they are intended to be equivalent in meaning and be open-ended in that an item or items following any one of these words is not meant to be an exhaustive listing of such item or items, or meant to be limited to only the listed item or items. It should be noted herein that any feature or component described in association with a specific embodiment may be used and implemented with any other embodiment unless clearly indicated otherwise.

It must also be noted that as used herein and in the appended claims, the singular forms “a,” “an,” and “the” include plural references unless the context dictates otherwise. Although any systems and methods similar or equivalent to those described herein can be used in the practice or testing of embodiments of the present disclosure, the preferred, systems and methods are now described.

In various embodiments, the present specification provides an integrated device comprising one or more varied types of electrodes mounted on, or within, an ultrasound probe for electro-diagnostic purposes. In embodiments, the ultrasound probe enables visualization of a patient's internal organs while the mounted electrodes may be used for one or more of: electrically stimulating the visualized internal organs (such as, but not limited to, nerves and nerve tracts) and recording electrical activity of said organs (such as, but not limited to, muscles and nerves).

In embodiments, the ultrasound probe is positioned within a hand-held probe body/housing which is coupled to an ultrasound machine comprising a display screen and a control board. In embodiments, the ultrasound probe further comprises an amplifier coupled to one or more electrodes and is configured to process electrical activity of the internal organ. Also, in embodiments, the ultrasound probe is in electrical communication with a pulse train generator configured to provide an electrical current of a predefined amplitude for stimulating the patient's internal organs, an ultrasound wave generator, at least one computing device/processor configured to process reflected ultrasound waves and a display. The at least one computing device/processor is coupled with memory for executing a plurality of programmatic instructions that generate an electrical pulse train which is delivered through the electrodes and ultrasound waves which are delivered through the ultrasound probe to the internal organs of a patient.

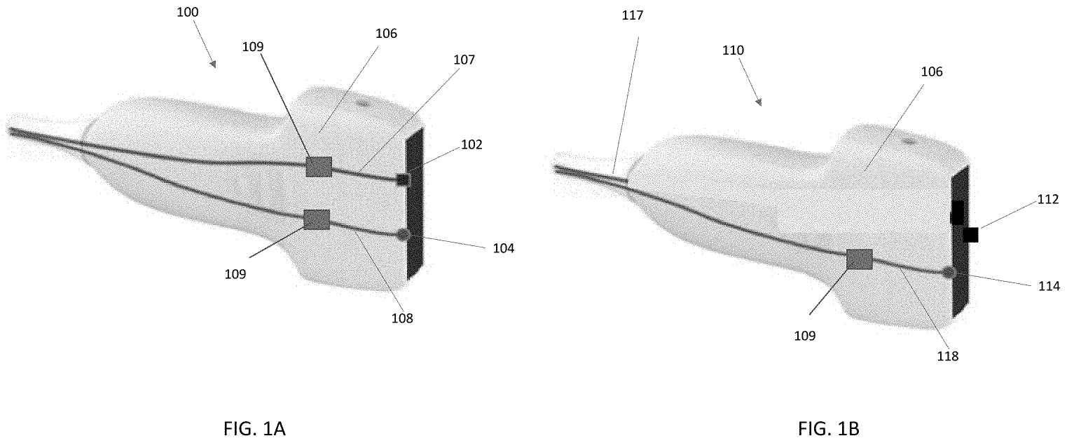

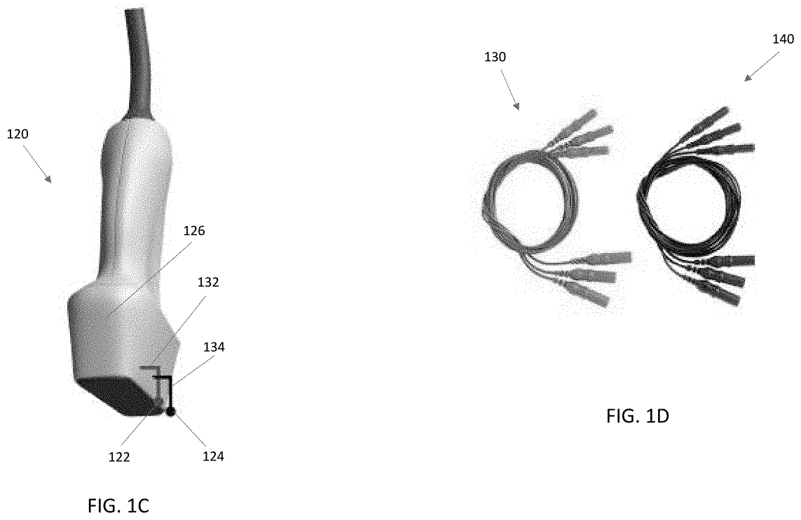

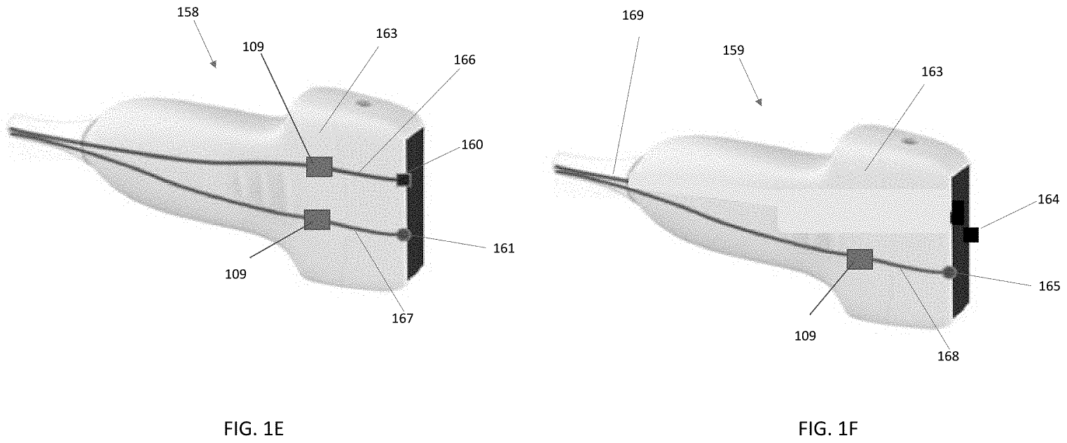

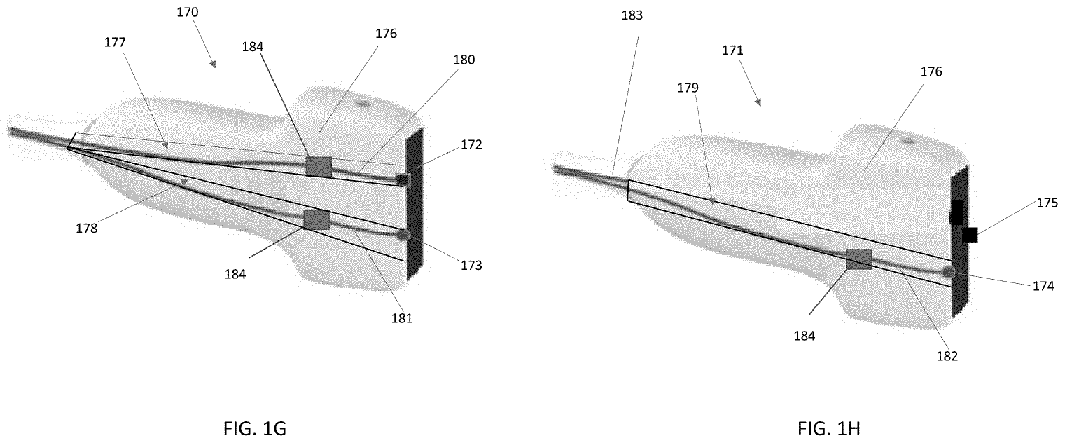

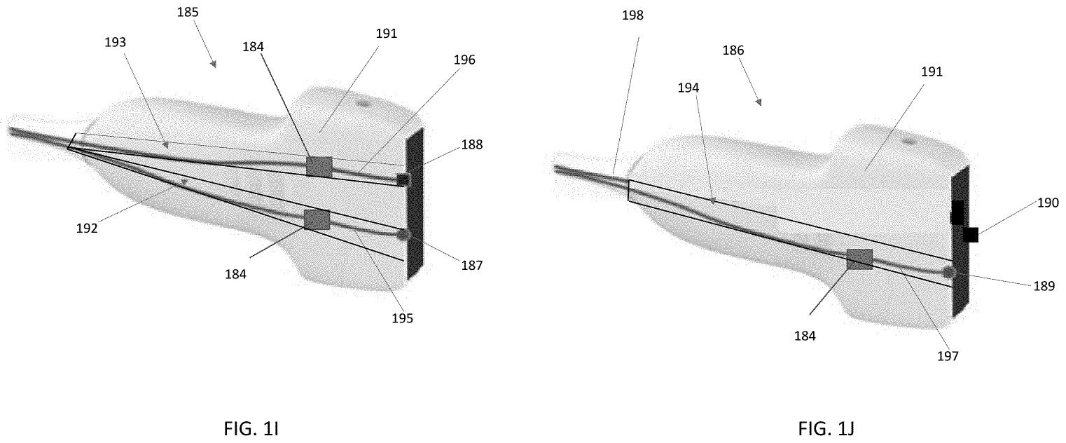

FIG. 1A illustrates two electrodes mounted on a same side of an ultrasound probe and FIG. 1B illustrates two electrodes mounted on different sides of an ultrasound probe, in accordance with embodiments of the present specification. Referring to both FIGS. 1A and 1B, device 100 comprises electrodes 102, 104 and device 110 comprises electrodes 112, 114 mounted on an ultrasound probe 106, wherein the electrodes 102, 104 are shown on the same side of the device 100 and the electrodes 112, 114 are shown on opposite sides of the device 110. It should be noted herein that electrodes 102 and 104 correspond to electrodes 112 and 114, respectively, depending on which embodiment is being referred to. Electrodes 102, 104 and 112, 114 are coupled with electrodiagnostic equipment (not shown in the figures) via connecting wires 107, 108 and 117, 118, respectively. It should be noted that wires 107, 108 correspond to wires 117, 118, depending upon which embodiment is being referred to, but that the wires are described with reference to the embodiments and generally.

In embodiments, the electrodes 102, 104 and 112, 114 are designed such that there is no electrical interference between the electrodes and the ultrasound probe 106, and such that the electrodes 102, 104 and 112, 114 do not block the ultrasound waves emanating from the probe 106. It should be noted that interference typically occurs when a portion of the wire or electrode obscures the probe element. In embodiments, interference may be reduced or eliminated by at least one of: reducing the size/thickness of the electrode and/or moving it away from the transmission elements of the probe. In an embodiment, the electrodes 102, 104 and 112, 114 are fabricated using thin wires 107, 108 and 117, 118, respectively, for eliminating interference. In embodiments, a wire that is greater than 0.02 mm thick has the potential to cause interference if the wire crosses the probe elements. In embodiments, the wires 107, 108 and 117, 118 are used to conduct a small amount of energy, and in embodiments a thickness of the wires 107, 108 and 117, 118 may be approximately as thin as 28 gauge or thicker. In a preferred embodiment, the thickness of the wires ranges between as thick as 12 gauge to 18 gauge for providing a desired design/functionality. In an embodiment, the electrodes 102, 104 and 112, 114 are placed on the probe 106 in a manner such that they are positioned away from an array of ultrasound elements within the probe 106, to eliminate any interference. In embodiments, the ultrasound probe 106 is positioned within a housing body and comprises a plurality of transmitting and receiving elements. The electrodes 102, 104 and 112, 114 are coupled to an exterior of the housing body and are placed such that a periphery of each of the electrodes 102, 104 and 112, 114 is positioned at least 2 mm from a periphery of each of the plurality of receiving elements to eliminate any interference.

FIG. 1C illustrates two electrodes mounted on a side of an ultrasound probe such that the electrodes are positioned away from an array of ultrasound elements within the probe, in accordance with an embodiment of the present specification. Device 120 comprises electrodes 122, 124 mounted on a side of an ultrasound probe 126, in order to prevent any interference with ultrasound elements within the probe. Electrodes 122, 124 are coupled with electrodiagnostic equipment (not shown in the figure) via connecting wires 132, 134 respectively.

FIG. 1E illustrates electrodes placed on a same side of an ultrasound probe, such that images of the electrodes are visible in the ultrasound image obtained via the probe, in accordance with an embodiment of the present specification. FIG. 1F illustrates electrodes placed on opposite sides of an ultrasound probe, such that images of the electrodes are visible in the ultrasound image obtained via the probe, in accordance with an embodiment of the present specification. Referring to FIGS. 1E and 1F, device 158 comprises electrodes 160, 161 and device 159 comprises electrodes 164, 165, which are respectively mounted on an ultrasound probe 163, wherein the electrodes 160, 161 are shown as placed on the same side of device 158 and the electrodes 164, 165 are shown as placed on opposite sides of device 159. Electrodes 160, 161 and 164, 165 are coupled with electrodiagnostic equipment (not shown in the figures) via connecting wires 166, 167 and 168, 169 respectively.

Referring to FIGS. 1E and 1F, in an embodiment, electrodes 160, 161 (placed on the same side of probe 163) and 164, 165 (placed on opposite sides of probe 163) are designed such that they interfere with the ultrasound waves emanating from the probe 163 so that the position of electrodes 160, 161 and 164, 165 can be visualized in the ultrasound image produced by using the probe 163. In embodiments, an ultrasound probe operates by sending mechanical vibrations (sound waves) into a patient's body and collecting the vibrations reflected by the internal organs of the body. If a material such as a gel or a large electrode having thick connecting wires is placed in the path of the reflected vibrations/sound waves, the material absorbs the reflected vibrations/sound waves, causing shadows in a resultant ultrasound image. In embodiments, an electrode larger than 0.5 mm will be visible in the ultrasound image. The extent of image obscured is proportional to the size of the electrode. In embodiments, the electrodes can be repositioned on the body as needed depending upon the anatomy of interest. In embodiments, the ultrasound probe 163 is positioned within a housing body and comprises a plurality of transmitting and receiving elements. The electrodes 166, 167 and 168, 169 are coupled to an exterior of the housing body and are placed such that a periphery of each of the electrodes 166, 167 and 168, 169 is positioned less than 2 mm from a periphery of each of the plurality of receiving elements to cause interference with ultrasound signals emanated by the probe 163. In embodiments where knowing the relative position of an electrode is important, it should be appreciated that the aforementioned size and positioning ranges are essential to the effective recordation of electrode positions using ultrasound. Alternatively, where it is important to not have the electrodes appear in the recorded ultrasound image, electrodes 166, 167 and 168, 169 are coupled to an exterior of the housing body and are placed such that a periphery of each of the electrodes 166, 167 and 168, 169 is positioned 2 mm or more from a periphery of each of the plurality of receiving elements to cause interference with ultrasound signals emanated by the probe 163.

In embodiments, the distance between each of the electrodes 160, 161 and between each of the electrodes 164, 165 placed on the probe 163 ranges from 10 mm to 25 mm. In an embodiment, a distance as specified by standards set in neuromuscular electro-diagnostics is maintained between each of the electrodes 160, 161 and between each of the electrodes 164, 165. In embodiments, the distance between each of the electrodes 160, 161 and between each of the electrodes 164, 165 varies based on a use of the electrodes combined with the ultrasound probe 163. In embodiments, the distance between each of the electrodes ranges between 5 mm and 30 mm. For example, in some embodiments, when the device 158 is used for adult patients the distance between the electrodes 160, 161 placed on the probe 163 is approximately 25 mm. For example, in some embodiments, when the device 159 is used for adult patients the distance between the electrodes 164, 165 placed on the probe 163 is approximately 25 mm. For example, in some embodiments, when the device 158 is used for pediatric patients the distance between the electrodes 160, 161 placed on the probe 163 is approximately 10 mm. For example, in some embodiments, when the device 159 is used for pediatric patients the distance between the electrodes 164, 165 placed on the probe 163 is approximately 10 mm.

In embodiments, the electrodes may be positioned on any side of the probe 106. The embodiment depicted in FIG. 1A shows the electrodes 102, 104 placed on the same side of the probe 106 for illustrative purposes only. The electrodes 112, 114 are placed on opposing sides of the probe 106, as shown in the embodiment depicted in FIG. 1B, without compromising the use and efficiency of the device 100. In still other embodiments, the electrodes may be placed on adjacent sides of the probe.

In embodiments, a length of the connecting wires 107, 108 and 117, 118 ranges from 100 mm to 2000 mm. In an embodiment, wherein the length of the connecting wires 107, 108 and 117, 118 is shorter than a required length, an extension cable may be used to connect the connecting wires 107, 108 and 117, 118, and thereby the corresponding electrodes, to the probe 106. In embodiments, the electrodes as well as the extension cables may be snapped to a cable of the ultrasound probe 106 for convenience. In embodiments, the extension cables may be standard off-the-shelf power extension cables. FIG. 1D illustrates exemplary extension cables 140, 150 that may be used with the connecting wires shown in FIGS. 1A-1C, in accordance with some embodiments of the present specification.

In embodiments, the electrodes 102, 104 and 112, 114 and 122, 124 may be sterile/disposable electrodes which are used during surgical procedures or non-sterile/reusable electrodes. In embodiments, reusable electrodes may be mounted directly on the probe 106 as shown in FIGS. 1A, 1B and 1C. In embodiments, reusable electrodes are mounted on ultrasound probes 106 by using a mechanical attachment 109 which allows the electrodes to be removed after use and then re-attached to the probe 106 when required. In embodiments, a plastic mold that is designed to fit around the probe or within a probe cover may be used and is configured to receive and hold in place the electrodes. In an embodiment, sterile/single-use electrodes may be coupled with an ultrasound probe by using any semi-permanent adhesive means, such as, but not limited to double sided tape. Once a procedure is complete, the sterile electrodes may be removed from the probe by removing the adhesive means and disposed of along with the adhesive means.

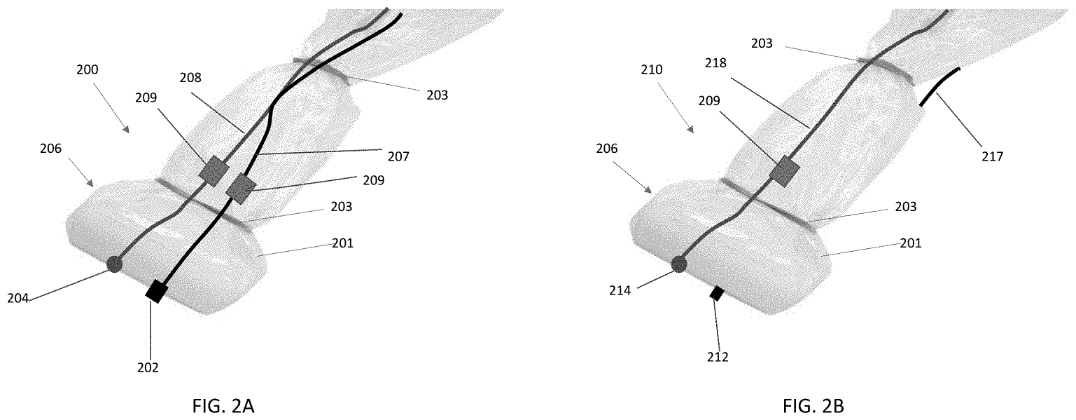

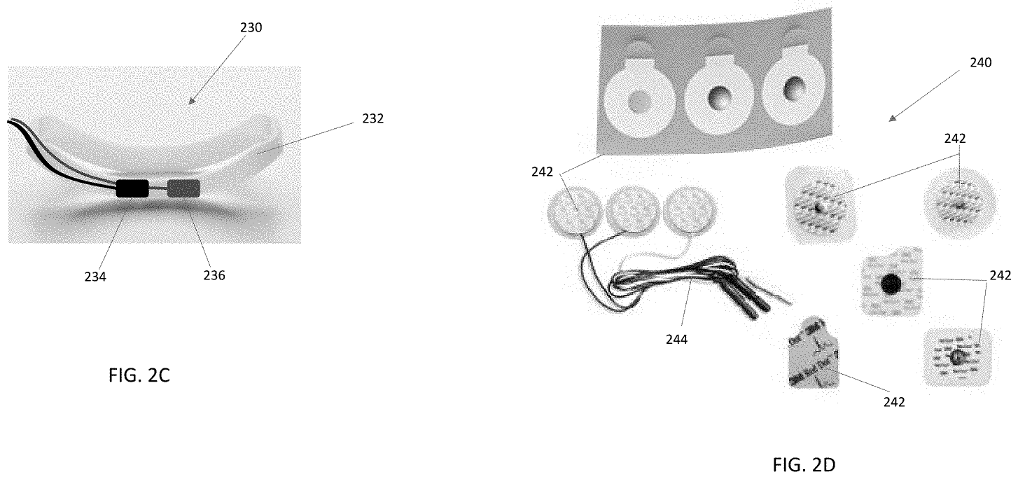

When used during surgery, where it is required that the electrodes are sterile, an ultrasound probe may be covered by using a sterile cover and the electrodes may then be mounted on the probe cover. FIG. 2A illustrates a device 200 comprising an ultrasound probe 206 covered by using a sterile cover 201 and with two electrodes 202, 204 mounted on the same side of the ultrasound probe 206, in accordance with an embodiment of the present specification. FIG. 2B illustrates a device 210 comprising an ultrasound probe 206 covered by using a sterile cover 201 and with two electrodes 212, 214 mounted on different sides of the ultrasound probe 206, in accordance with an embodiment of the present specification. Electrodes 202, 204 and 212, 214 are coupled with electrodiagnostic equipment (not shown in the figures) via connecting wires 207, 208 and 217, 218, respectively. In embodiments, the cover 202 is held in place over the probe 206 by using a fastening mechanism 203. In some embodiments, the fastening mechanism comprises elastics or rubber bands 203. In an embodiment, reusable electrodes are mounted on covered ultrasound probes 206 by using a mechanical attachment 209 which allows the electrodes to be removed from the cover 201 after use and then re-attached to a probe or covered probe when required. In an embodiment, where disposable electrodes are used, the mechanical attachment 209 may be any semi-permanent adhesive means, such as, but not limited to adhesive tape.

FIG. 1G illustrates electrodes placed on a same side of an ultrasound probe, such that there is no interference between the electrodes and the signal of the probe, wherein the electrodes are placed within a channel molded on the probe body, in accordance with an embodiment of the present specification. FIG. 1H illustrates electrodes placed on opposite sides of an ultrasound probe, such that there is no interference between the electrodes and the signal of the probe, wherein the electrodes are placed within a channel molded on the probe body, in accordance with an embodiment of the present specification. As shown in the figures, device 170 comprises electrodes 172, 173 mounted on an ultrasound probed 176, wherein the wherein the electrodes 172, 173 are positioned on the same side of the device 170. Also as shown, device 171 comprises electrodes 174, 175 mounted on an ultrasound probe 176, wherein the electrodes 174, 175 are positioned on opposite sides of the device 171.

In an embodiment, as shown in FIG. 1G, channels 177, 178 are formed within the body of probe 176, for housing each of the electrodes 172, 173, respectively. In an embodiment, as shown in FIG. 1H, a channel 179 is formed within one side of the body of probe 176, for housing electrode 174, and a second channel (not visible in the figure) is formed within an opposing side of the body of probe 176 for housing electrode 175. Each of the electrodes 172, 173 and 174, 175 are coupled with electrodiagnostic equipment (not shown in the figures) via connecting wires 180, 181 and 182, 183, respectively. In an embodiment, the channels are molded into the probe body as grooves configured to receive and guide the electrodes along with corresponding connecting wires for correctly positioning the electrodes. In embodiments, the electrodes 172, 173 and 174, 175 are designed such that there is no electrical interference between the electrodes and the ultrasound probe 176, and such that the electrodes 172, 173 and 174, 175 do not block the ultrasound waves emanating from the probe 176. In an embodiment, the electrodes 172, 173 and 174, 175 are fabricated using thin wires for eliminating interference. In embodiments, a wire that is greater than 0.02 mm thick has the potential to cause interference if the wire crosses the probe elements. In embodiments, the ultrasound probe conducts electrical signals that may potentially interfere with the signals that are being recorded by the attached recording electrodes. In this case, shielding may be employed, for example, by shielding the recording electrodes or wires and/or shielding the probe and/or probe cable. In embodiments, reusable electrodes 172, 173 and 174, 175 are mounted within respective channels on ultrasound probes 176 by using a mechanical attachment 184 which allows the electrodes to be removed after use and then re-attached to the probe 176 when required. In embodiments, the ultrasound probe 176 is positioned within a housing body, and comprises a plurality of transmitting and receiving elements. The above said channels are molded on an outer surface of said housing. The electrodes 172, 173 and 174, 175 are coupled to an exterior of the housing body, and are placed such that a periphery of each of the electrodes 172, 173 and 174, 175 is positioned at least 2 mm from a periphery of each of the plurality of receiving elements to eliminate any interference.

FIG. 1I illustrates electrodes placed on a same side of an ultrasound probe, such that images of the electrodes are visible in the ultrasound image obtained via the probe, wherein the electrodes are placed within a channel molded on the probe body, in accordance with an embodiment of the present specification. FIG. 1J illustrates electrodes placed on opposite sides of an ultrasound probe, such that images of the electrodes are visible in the ultrasound image obtained via the probe, wherein the electrodes are placed within a channel molded on the probe body, in accordance with an embodiment of the present specification. As shown in the figures, device 185 comprises electrodes 187, 188 mounted on an ultrasound probe 191, wherein the electrodes 187, 188 are positioned on the same side of the device 185. Also as shown in the figures, device 186 comprises electrodes 189, 190 mounted on an ultrasound probe 191, wherein the electrodes 189, 190 are positioned on opposite sides of the device 186.

In an embodiment, as shown in FIG. 1I, channels 192, 193 are formed within the body of probe 191, for housing the electrodes 187, 188, respectively. In the embodiment, shown in FIG. 1J a channel 194 is formed on one side of the body of probe 191 for housing the electrode 189, and a second channel (not visible in the figure) is formed on an opposing side of the body of probe 191 for housing the electrode 190. Electrodes 187, 188 and 189, 190 are coupled with electrodiagnostic equipment (not shown in the figures) via connecting wires 195, 196 and 197, 198, respectively. Referring to FIGS. 1I and 1J, in an embodiment, electrodes 187, 188 (placed on the same side of probe 191) and 189, 190 (placed on opposite sides of probe 191) are designed to interfere with the ultrasound waves emanating from the probe 191 so that the position of electrodes 187, 188 and 189, 190 can be visualized in the ultrasound image produced by using the probe 191. In an embodiment, the channels are molded onto the probe body as grooves configured to receive and guide the electrodes along with corresponding connecting wires for correctly positioning the electrodes. In embodiments, reusable electrodes 187, 188 and 189, 190 are mounted in respective channels on ultrasound probes 191 by using a mechanical attachment 184 which allows the electrodes to be removed after use and then re-attached to the probe 191 when required. In embodiments, reusable electrodes 187, 188 and 189, 190 are mounted in respective channels on ultrasound probes 191 by using a mechanical attachment 184 which allows the electrodes to be removed after use and then re-attached to the probe 191 when required. In embodiments, the ultrasound probe 191 is positioned within a housing body and comprises a plurality of transmitting and receiving elements. The above said channels are molded on an outer surface of said housing. The electrodes 187, 188 and 189, 190 are coupled to an exterior of the housing body and are placed such that a periphery of each of the electrodes 187, 188 and 189, 190 is positioned at least 0.5 mm at from a periphery of each of the plurality of receiving elements to cause interference with ultrasound signals emanated by the probe 191.

FIG. 2C illustrates a mechanical attachment holding a pair of electrodes that may be removably attached to an ultrasound probe, in accordance with an embodiment of the present specification. Attachment 230 comprises a portion 232 which may be snapped on to an ultrasound probe. Portion 232 is shaped and sized to fit onto any standard sized ultrasound probe. Electrodes 234, 236 are coupled with the portion 232, such that when the portion 232 is snapped on an ultrasound probe, the electrodes 234, 236 are coupled with the probe as shown in FIG. 1A. In an embodiment, portion 232 is made of plastic.

FIG. 2D illustrates a plurality of electrodes that include an adhesive back for attachment with an ultrasound probe, in accordance with an embodiment of the present specification. Electrodes 240 include an adhesive on one side which is covered with a protective backing 242. For use, the protective backing 242 may be peeled off and the electrodes 240 may be stuck onto an ultrasound probe by bringing the adhesive side of the electrodes 240 in contact with a surface of the probe, while an opposing conductive side of the electrodes 240 faces outwards towards a user. As shown electrodes 240 may be provided with connecting wires 244. Once a procedure is complete, the sterile electrodes may be removed from the covered probe by removing the adhesive means and disposed of along with the adhesive means or, the entire cover with attached sterile/single-use electrodes may be removed from the probe together and disposed of.

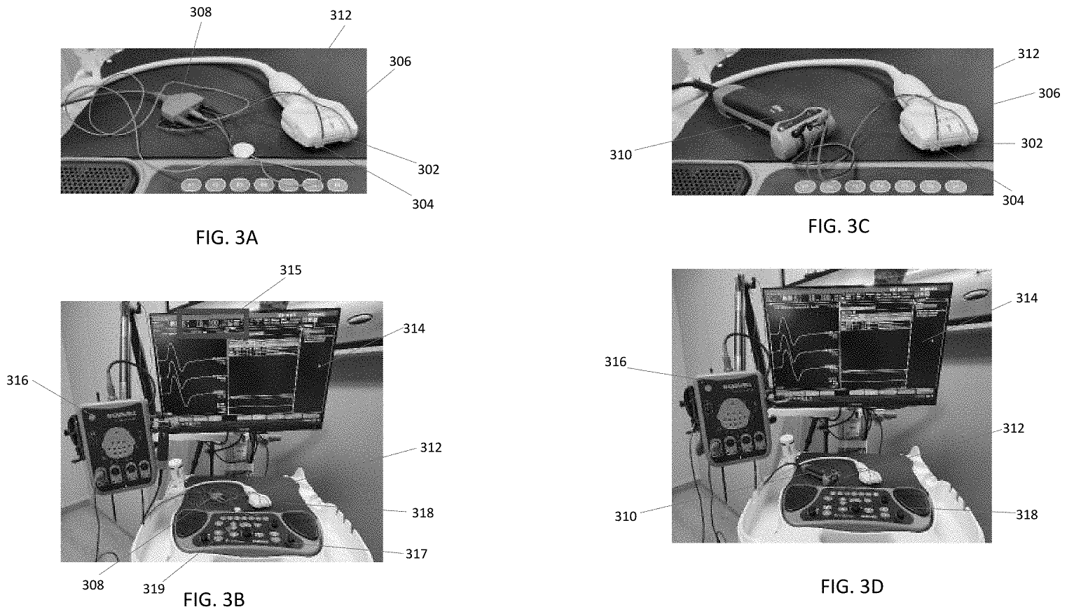

FIG. 3A illustrates electrodes coupled with an ultrasound probe, wherein the electrodes are attached to a connector, in accordance with an embodiment of the present specification. FIG. 3B illustrates a display of an electro-diagnostic equipment for viewing the electrical activity recorded by the electrodes shown in FIG. 3A. Referring to FIGS. 3A and 3B, electrodes 302, 304 are coupled with an ultrasound probe 306. In FIG. 3A the electrodes 302, 304 are attached to a connector 308 which, in turn, is connected to the amplifier 316 shown in FIG. 3B. In embodiments, connector 308 is a standard connector for an amplifier or stimulator. Electrodes can be connected directly to the amplifier or stimulator or via an extension cable when needed. The electrodes 302, 304 are used to record the electrical activity within an organ. FIG. 3B illustrates connector 308 connected with the pair of electrodes 302, 304 and connected to amplifier 316. Referring to FIGS. 3A and 3B, electrodes 302, 304 are coupled with the ultrasound probe 306, and are also connected to the connector 308 via connecting wires 312. The connector 308 is connected to amplifier 316. The electrical activity recorded by the electrodes 302, 304 is displayed on a screen 314 of the equipment. A section 315 on the screen 314 displays an intensity and other parameters of the stimuli being provided to a patient's body via the electrodes 302, 304. A control board 318, connected to the screen 314, may be used to control the functionality of the probe 306 and the electrodes 302, 304, such as, but not limited to the intensity of stimuli being provided, by using a plurality of knobs 317 and buttons 319 provided on the control board 318, a touch screen display, or a conventional computer display controlled by a keyboard and/or mouse. In embodiments, the control board can be used to control all ultrasound, stimulator, and amplifier parameters.

FIG. 3C illustrates the electrodes 302, 304 connected to a stimulator 310 for electrically stimulating (providing electrical pulses of predefined amplitude) an organ. In an embodiment, the present specification may be coupled with or used with electrical, constant current stimulators, or constant voltage stimulators. In embodiments, the electrodes 302, 304 have standard Touch Proof or DIN connectors 312 for connecting with either the connector 308 or the electrical stimulator 310 on any electrodiagnostic equipment supporting standard connectors. FIG. 3D is an illustration showing the stimulator 310 connected with the pair of electrodes and connected to an amplifier 316. Referring to FIGS. 3C and 3D, electrodes 302, 304 are coupled with the ultrasound probe 306, and are also connected to the stimulator 310 via connecting wires 312. The electrodes 302, 304 in FIG. 3D are used to provide electrical stimulations to an organ which is simultaneously viewed internally by using the ultrasound probe 306. In embodiments, parameters such as, but not limited to an amplitude of the electrical stimulations, stimuli intensity, stimuli duration and stimulation frequency may be adjusted by using the control board 318 and may be viewed via the screen 314 connected to the control board 318.

In various embodiments, the device of the present specification combines electrodiagnostic technology with ultrasound technology and may be used in clinical use cases such as, but not limited to: identifying the sural nerve in a patient's leg and stimulating said nerve at an optimal position; identifying a patient's median nerve and recording orthodromic sensory nerve conduction (SNC) from the identified nerve; performing a nerve graft by identifying a nerve, obtaining fascicular information of the corresponding patient, and stimulating the nerve/fascicles to confirm functionality of the nerve; and, identifying structure and stimulating to verify nerve functionality during cranial surgery.

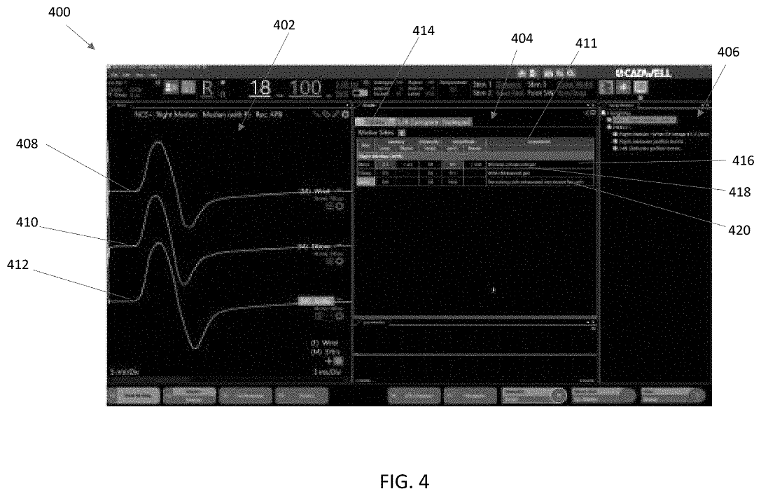

FIG. 4 illustrates a graphical user interface display showing waveforms recorded with recording/stimulating electrodes placed on a patient's arm, in accordance with an embodiment of the present specification. Display 400 comprises: a first section 402 for displaying waveforms representing electrical activity in a patient's organ being stimulated/recorded with electrodes; a second section 404 for providing information regarding the one or more portions of the patient's organ being stimulated; and a third section 406 for providing a navigational tool for accessing information regarding the specific organ portions. In embodiments, first section 402 displays: a first plot 408 of the electrical activity recorded by one or more stimulating electrodes positioned on the patient's wrist; a second plot 410 of the electrical activity recorded by one or more stimulating electrodes positioned on the patient's elbow; and a third plot 412 of the electrical activity recorded by one or more recording electrodes positioned on the patient's axilla/armpit. Second section 404 is shown as providing information in table 414 about the right median (APB) nerve of the patient. Row 416 of table 414 display values for a latency, intensity and amplitude corresponding to the first plot 408 of the electrical activity recorded by one or more stimulating electrodes positioned on the patient's wrist, which, in an embodiment are 3.7 ms, 16 mA, and 8.9 mV, respectively. Since, in this case, the latency and amplitude lie outside a predefined normal range, the values are highlighted. Table 414 comprises a comments column 411. Row 416, and column 411, which is a comments section, also displays that the electrical activity recorded by one or more stimulating electrodes positioned on the patient's wrist is obtained without the use of ultrasound gel, as the conducting gel may interfere with the electrical activity being recorded by the electrodes.

Row 418 of table 414 displays values for a latency, intensity and amplitude corresponding to the second plot 410 of the electrical activity recorded by one or more stimulating electrodes positioned on the patient's elbow, which, in an embodiment are 3.5 ms, 16 mA, and 9.1 mV, respectively. Row 418 and column 411, which is a comments section, displays that the electrical activity recorded by one or more stimulating electrodes positioned on the patient's elbow is obtained by using an ultrasound gel applied between the electrodes and the patient's skin.

Row 420 of table 414 displays values for a latency, intensity and amplitude corresponding to the third plot 412 of the electrical activity recorded by one or more recording electrodes positioned on the patient's axilla/armpit, which, in an embodiment are 3.6 ms, 18 mA, and 10.4 mV respectively. Row 420 and column 411, which is a comments section, also displays that the electrical activity recorded by one or more recording electrodes positioned on the patient's axilla/armpit is obtained without the use of ultrasound gel. Third plot 412 shows a larger response since the electrode recording surface is smaller. As is known, a large electrode recording surface sums/averages electrical activity from a larger region, which typically results in a smaller response, as compared to the response of an electrode having a smaller recording surface.

In various embodiments, a location and a polarity of an electrode mounted on an ultrasound probe is based upon how the electrode is mounted on said probe. Typically, ultrasound probes are provided with directional markings enabling a physician to place the probe on a patient's body in a desired direction. In embodiments, the electrodes mounted on an ultrasound probe may be aligned with the direction markings on the probe to obtain a desired direction/polarity with respect to the electrodes.

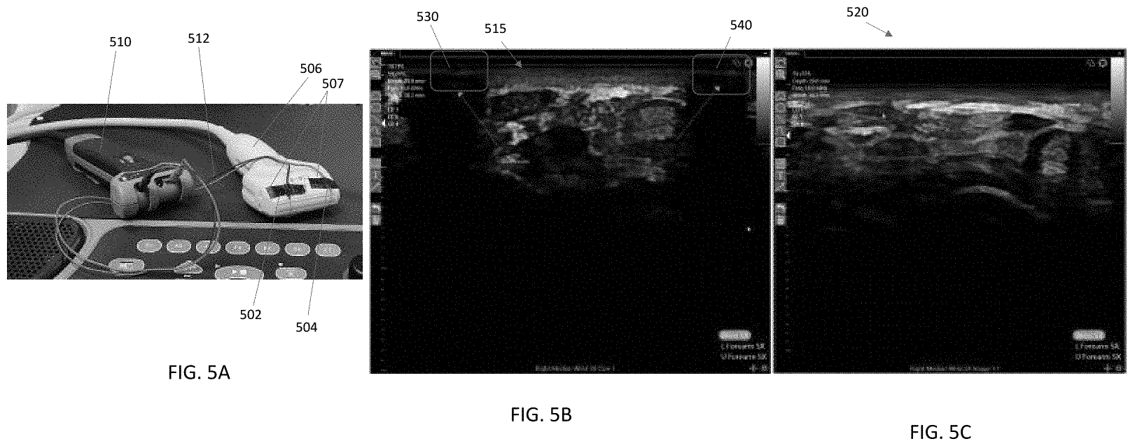

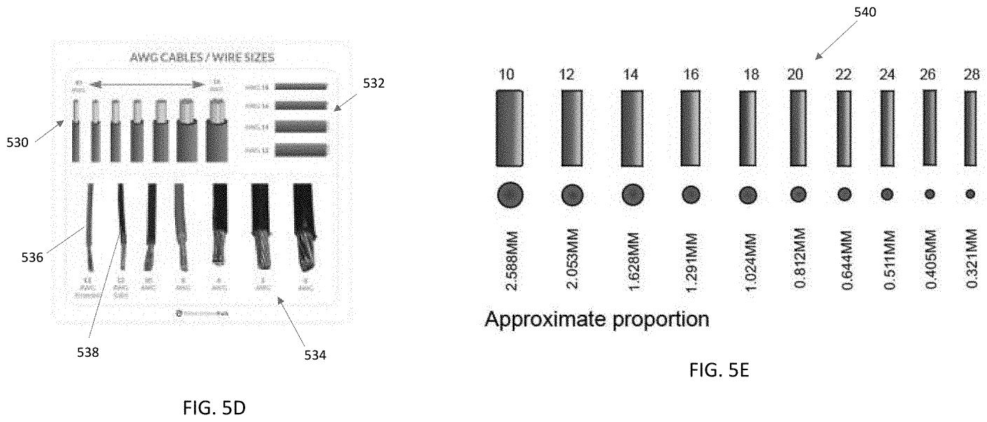

FIG. 5A illustrates electrodes coupled with an ultrasound probe, wherein the electrodes are connected to a stimulator, in accordance with an embodiment of the present specification. As shown in FIG. 5A electrodes 502, 504 are adhered to an ultrasound probe 506 by using strips of adhesive tape 507. The electrodes 502, 504 are also connected to a stimulator 510 via connecting wires 512. The electrodes 502, 504 are used to provide electrical stimulation to an organ which is simultaneously viewed internally by using the ultrasound probe 506. FIG. 5B illustrates an ultrasound image of a patient's internal organ obtained by using the ultrasound probe coupled with electrodes shown in FIG. 5A. As shown in FIG. 5B, the electrodes 502, 504 each create a shadow 530, and 540 respectively in the ultrasound image 515. The shadows are caused by the electrodes 502, 504 disrupting or absorbing the sound waves emanated by the ultrasound probe 506 and reflected by internal organs of a patient receiving the ultrasound. Hence, the shadow observed on the ultrasound image 515 is directly proportional to the footprint of the electrodes 502, 504 and their respective connecting wires 512. In embodiments, shadows 530, 540 may be reduced by using thinner and smaller strips of adhesive tape 507 for coupling the electrodes 502, 504 with the probe 506. In other embodiments, shadows 530, 540 may be reduced or eliminated by offsetting the electrode from the other elements. In still other embodiments, electrodes may be placed on a mount that is parallel and a distance away from the probe elements may reduce and/or eliminate interference. The shadows 530, 540 may be used to indicate which electrode is G1/G2 and which electrode is the anode or cathode by using different shapes/sizes of mounting means for mounting the electrodes 502, 504 on the probe 506.

FIG. 5C illustrates an ultrasound image 520 of a patient's internal organ obtained by using an ultrasound probe coupled with electrodes having thin connecting wires. The thin wires do not interfere with the ultrasound image while providing the same recording/stimulation characteristics as shown in FIG. 5B. Hence, the image 520 does not contain shadows 530, 540 that were visible in the image 515.

FIG. 5D illustrates connecting wires of varying thickness that may be used with electrodes coupled with an ultrasound probe, in accordance with embodiments of the present specification. In embodiments, connecting wires having standard American Wire Gauge (AWG) sizes may be used. In FIG. 5D, section 530 illustrates wires having a thickness ranging from 40 AWG (being the thinnest) to 18 AWG (being the thickest). Section 532 illustrates wires having a thickness ranging from 18 AWG (being the thinnest) to 12 AWG (being the thickest). Section 534 illustrates wires having a thickness ranging from 12 AWG (being the thinnest) to 0 AWG (being the thickest), wherein wire 538 is a solid 12 AWG wire and wire 536 is a stranded 12 AWG wire. Any of the wires shown in FIG. 5D may be used with electrodes in various embodiments of the present specification. FIG. 5E illustrates a plurality of differently sized wires that may be used with electrodes coupled with an ultrasound probe, in accordance with embodiments of the present specification. As shown in FIG. 5E wires 540 ranging from 10 AWG having a thickness of 2.588 mm to 28 AWG having a thickness of 0.321 mm may be coupled with electrodes for stimulating a patient's internal organs in conjunction with an ultrasound probe.

The above examples are merely illustrative of the many applications of the system of present specification. Although only a few embodiments of the present invention have been described herein, it should be understood that the present invention might be embodied in many other specific forms without departing from the spirit or scope of the invention. Therefore, the present examples and embodiments are to be considered as illustrative and not restrictive, and the invention may be modified within the scope of the appended claims.

What is claimed is:

1. A probe configured to direct an electrical stimulation to an internal organ of a patient while concurrently directing ultrasound energy to said internal organ of the patient, wherein the handheld probe is in electrical communication with a pulse train generator, an ultrasound wave generator, at least one processor configured to process reflected ultrasound waves and a display, the handheld probe comprising:
a housing body;
an ultrasound probe positioned within the housing body, wherein the ultrasound probe comprises a plurality of transmitting elements and a plurality of receiving elements; and
one or more electrodes coupled to an exterior of the housing body, wherein each of the one or more electrodes is positioned such that a periphery of each of the one or more electrodes is positioned less than 2 mm from a periphery of each of the plurality of receiving elements.

2. The probe of claim 1, wherein the pulse train generator is configured to provide an electric current of a predefined amplitude for stimulating the internal organ.

3. The probe of claim 1, wherein the one or more electrodes are single-use disposable electrodes.

4. The probe of claim 3, wherein the one or more single-use electrodes are coupled with the housing body using adhesive or a molded housing.

5. The probe of claim 1, wherein the one or more electrodes are reusable electrodes.

6. The probe of claim 5, wherein the one or more reusable electrodes are coupled with the housing body using adhesive or a molded housing.

7. The probe of claim 1, wherein the one or more electrodes are coupled to the pulse train generator using connecting wires having a thickness ranging from 1 AWG to 40 AWG, and preferably 10 AWG to 28 AWG.

8. The probe of claim 7, wherein the connecting wires comprise touchproof or DIN connecting wires.

9. The probe of claim 1, wherein a distance between the one or more electrodes is in a range of 10 mm to 25 mm.

10. The probe of claim 1, wherein the ultrasound probe is coupled to an ultrasound machine comprising a display screen and a control board.

11. The probe of claim 1, further comprising an amplifier coupled to the one or more electrodes and configured to process electrical activity of the internal organ.

12. A probe configured to direct an electrical stimulation to an internal organ of a patient while concurrently directing ultrasound energy to said internal organ of the patient, wherein the handheld probe is in electrical communication with a pulse train generator, an ultrasound wave generator, at least one processor configured to process reflected ultrasound waves and a display, the handheld probe comprising:
a housing body;
an ultrasound probe positioned within the housing body, wherein the ultrasound probe comprises a plurality of transmitting elements and a plurality of receiving elements; and
one or more electrodes coupled to an exterior of the housing body, wherein each of the one or more electrodes is positioned such that a periphery of each of the one or more electrodes is positioned 2 mm or more from a periphery of each of the plurality of receiving elements.

13. The probe of claim 12, wherein the pulse train generator is configured to provide an electric current of a predefined amplitude for stimulating the internal organ.

14. The probe of claim 12, wherein the one or more electrodes are single-use disposable electrodes.

15. The probe of claim 14, wherein the one or more single-use electrodes are coupled with the housing body using adhesive or a molded housing.

16. The probe of claim 12, wherein the one or more electrodes are reusable electrodes.

17. The probe of claim 16, wherein the one or more reusable electrodes are coupled with the housing body using adhesive or a molded housing.

18. The probe of claim 12, wherein the one or more electrodes are coupled to the pulse train generator using connecting wires having a thickness ranging from 1 AWG to 40 AWG, and preferably 10 AWG to 28 AWG.

19. The probe of claim 18, wherein the connecting wires comprise touchproof or DIN connecting wires.

20. The probe of claim 12, wherein a distance between the one or more electrodes is in a range of 10 mm to 25 mm.

21. The probe of claim 12, wherein the ultrasound probe is coupled to an ultrasound machine comprising a display screen and a control board.

22. The probe of claim 12, further comprising an amplifier coupled to the one or more electrodes and configured to process electrical activity of the internal organ.
